(19) United States
(12) Patent Application Publication
(10) Pub. No.: US 2022/0370511 A1
(43) Pub. Date: Nov. 24, 2022

(54) METHODS AND COMPOSITIONS FOR ENHANCING OVERALL HEALTH AND LONGEVITY IN MAMMALS

(71) Applicant: MYOS CORP., Cedar Knolls, NJ (US)

(72) Inventors: Neerav D. PADLIYA, Cedar Knolls, NJ (US); Maghsoud DARIANI, Cedar Knolls, NJ (US)

(21) Appl. No.: 17/770,389

(22) PCT Filed: Nov. 5, 2020
(86) PCT No.: PCT/US2020/059030

§ 371 Date: Apr. 20, 2022

Related U.S. Application Data
(60) Provisional application No. 62/930,704, filed on Nov. 5, 2019.

(51) Int. Cl. A61K 35/57; A61K 31/706; A61K 31/085

(52) U.S. Cl. CPC A61K 35/57; A61K 31/706; A61K 31/085

(57) ABSTRACT

Compositions containing egg yolk powder and a NAD+ precursor and/or pterostilbene and/or an agent promoting mitochondrial rejuvenation for use in enhancing overall health and longevity in mammals are provided.

This patent application claims the benefit of priority from U.S. Provisional Application Ser. No. 62/930,704 filed Nov. 5, 2029, the teachings of which are incorporated by reference in their entirety herein.

### FIELD OF THE INVENTION

Embodiments of the present invention are generally related to methods of enhancing overall health and longevity in a mammal using avian egg powder in combination with a NAD+ precursor and/or pterostilbene. More specifically, embodiments of the present invention relate to a method of enhancing longevity by administering to a mammal a composition containing a powder derived from an avian egg in combination with a NAD+ precursor and/or pterostilbene in an effective amount, and/or the like.

### BACKGROUND

The protein known as avian follistatin, and in particular, chicken follistatin, has been identified as a very beneficial protein for human consumption as it pertains to the health of lean muscle tissue. Such benefits are described in part in commonly owned United States Patent Application Publication No. 2007/0275036, the disclosure of which is incorporated herein by reference in its entirety (hereinafter “Green Application”). The Green Application additionally discloses numerous products made using dried fertilized egg, and the muscle enhancing benefits thereof.

As disclosed in the Green Application, one embodiment of an egg-based product may be derived by extracting follistatin from the membrane of a fertilized chicken egg and processing the same.

U.S. Pat. No. 8,815,320 and Published U.S. Application No. 2018/0368425 disclose a process for producing a composition containing active follistatin for increasing muscle mass.

Nicotinamide adenine dinucleotide (NAD⁺) is a critical co-substrate for enzymes involved in the beneficial effects of regular calorie restriction on healthspan. NAD+ is found in every cell of the body and is involved in hundreds of metabolic processes. However, NAD+ levels decline with age. The impact of supplements designed to increase NAD⁺ on the aging process is still being studied. For example, the use of NAD+ precursors to augment NAD⁺ bioavailability has been proposed as a strategy for improving cardiovascular and other physiological functions with aging in humans. A 2×6-week randomized, double-blind, placebo-controlled, crossover clinical trial showed that chronic supplementation with the NAD+ precursor vitamin, nicotinamide riboside (NR), was well tolerated and effectively stimulated NAD⁺ metabolism in healthy middle-aged and older adults (Martins et al. Nature Communications 2018 9:1286). However, scientific evidence for proposed uses of NAD+ precursors in anti-aging, high cholesterol, obesity, and other conditions is suggested to be lacking. See webmd.com/vitamins/ai/ ingredientmono-1560/nicotinamide-riboside.

Pterostilbene(trans-3,5-dimethoxy-4-hydroxystilbene) is a natural dietary compound and the primary antioxidant component of blueberries. It has increased bioavailability in comparison to other stilbene compounds, which may enhance its dietary benefit and possibly contribute to a valuable clinical effect. Multiple studies have demonstrated the antioxidant activity of pterostilbene in both in vitro and in vivo models illustrating both preventative and therapeutic benefits. The antioxidant activity of pterostilbene has been implicated in anticarcinogenesis, modulation of neurological disease, anti-inflammation, attenuation of vascular disease, and amelioration of diabetes. See McCormack and McFadden Oxidative Medicine and Cellular Longevity 2013 Article ID 575482). However, no clinical evidence supports the use of pterostilbene for any of these conditions and the Food and Drug Administration has not approved pterostilbene for any medical purpose or health claim.

### SUMMARY

An aspect of the present invention relates to a method for enhancing overall health and longevity in a mammal. The method comprises administering to the mammal a composition comprising egg yolk powder in combination with an NAD+ precursor and/or pterostilbene.

In one nonlimiting embodiment, the composition comprises a fertilized egg yolk derived product.

In one nonlimiting embodiment, the fertilized egg yolk derived product is FORTETROPIN.

In one nonlimiting embodiment, the mammal is administered a composition comprising both the egg yolk powder and the NAD+ precursor and/or pterostilbene.

In one nonlimiting embodiment, the NAD+ precursor is nicotinamide riboside.

Another aspect of the present invention relates to a composition for enhancing overall health and longevity in mammals. The composition of this present invention comprises egg yolk powder and an NAD+ precursor and/or pterostilbene.

In one nonlimiting embodiment, the composition comprises a fertilized egg yolk derived product.

In one nonlimiting embodiment, the fertilized egg yolk derived product is FORTETROPIN.

In one nonlimiting embodiment, the NAD+ precursor is nicotinamide riboside.

### DETAILED DESCRIPTION

It is understood that the embodiments of the present invention are not limited to the particular methodologies, protocols and the like, described herein as they may vary. It is also to be understood the terminology used herein is used for the purpose of describing particular embodiments only and not intended to limit the scope of the present invention.

Unless defined otherwise, all technical and scientific terms used herein have the same meaning as commonly understood by one of ordinary skilled in the art to which this invention belongs.

Although mitochondria, organelles that serve as the powerhouses of cells serve an important function in all mammalian cell types, they serve a profoundly important function in skeletal muscle cells where they exist in high densities due to the high energy requirements of these muscle cells. Not only is maintaining mitochondrial health vital to musculoskeletal health, it is also vital to both cognitive and metabolic health. Improvements in muscle mass lead to lower incidences of fall injury, improved mobility and improved cancer survival. Oxidative stress also plays a broad role in many diseases ranging from chronic obstructive pulmonary disease (COPD) to rheumatoid arthritis to cancer. Thus, antioxidants play an important role in providing protection from diseases that can lead to decreased longevity.

It has now been found that taking an egg powder based composition and adding an NAD+ precursor and/or pterostilbene and/or an agent that promotes mitochondrial rejuvenation thereto results in unexpected benefits in enhancing overall health and longevity in mammals. Thus, disclosed herein are methods for enhancing overall health and longevity in mammals via administration of a composition comprising egg yolk powder in combination with an NAD+ precursor and/or pterostilbene and/or an agent that promotes mitochondrial rejuvenation and compositions comprising both egg yolk powder and nicotinamide riboside and/or pterostilbene and/or an agent that promotes mitochondrial rejuvenation. Administration of a composition comprising fertilized egg yolk powder in combination with an NAD+ precursor and/or pterostilbene and/or an agent that promotes mitochondrial rejuvenation enhances overall health and longevity in mammals through a holistic, multifaceted approach that serves to promote mitochondrial health while also leading to gains in muscle thickness and providing protection against oxidative damage.

By “enhancing overall health and longevity” in a mammal it is meant to include, but is not limited to, any reduction in chronic illnesses, an increase in life span, improvement in mobility and balance and/or improved cognitive and metabolic health as compared to a mammal not receiving a composition comprising fertilized egg yolk powder in combination with an NAD+ precursor and/or pterostilbene.

By “mammals” it is meant to include, but is not limited to, humans, apes, monkeys, cows, horses, sheep, goats, dogs, cats, mice, rats, and transgenic species thereof.

In one nonlimiting embodiment, the mammal is a human.

Compositions administered in accordance with the present invention comprise egg yolk powder or one or more proteins and/or lipids derived from egg yolk, which are effective in enhancing muscle growth and/or slowing muscle degeneration in mammals.

In one nonlimiting embodiment, the process for producing the egg yolk powder, and the egg yolk powder itself, are free from added chemical preservatives. In one nonlimiting embodiment, the process for producing the egg yolk powder, and the egg yolk powder, respectively, essentially consist of the natural components of the biological source or starting material. In one nonlimiting embodiment, the biological source is egg and its components, preferably egg yolk optionally including the white of egg, only subject to the physical treatment steps of the process. In the alternative to egg yolk, white of egg, which is also called egg white or egg albumen, and whole egg can be subjected to the steps of the process. In one nonlimiting embodiment, the egg is avian egg. In one nonlimiting embodiment, the avian egg is a fertilized avian egg. In one nonlimiting embodiment, the fertilized avian egg is from a domestic fowl selected from a turkey, chicken, duck, goose and ostrich. In another nonlimiting embodiment, the biological source is raw animal blood serum.

In one nonlimiting embodiment, fertilized avian eggs are obtained from hens. In one nonlimiting embodiment, the fertilized eggs used are collected within 24 hours post-lay. The eggs are collected and stored in a refrigerator or cold room (at or lower than 4° C. (40° F.)) until they are ready to be processed. The fertilized eggs are used within 3 weeks.

In one nonlimiting embodiment, the egg yolk powder administered is FORTETROPIN. FORTETROPIN is a fertilized egg yolk derived product used as a dietary and nutritional supplement (MYOS RENS TECHNOLOGY INC., Cedar Knolls, N.J.). Methods for production of FORTETROPIN, an egg yolk powder derived from raw liquid egg yolk, raw liquid whole egg or raw liquid egg white originating from fertilized avian egg and subjected to a step of preservation comprising high pressure treatment and/or pulsed electric field treatment are disclosed in U.S. Pat. Nos. 8,815,320, and 10,165,785, teachings of each which are herein incorporated by reference in their entirety.

The dietary supplement FORTETROPIN, derived from fertilized egg yolk and processed using technologies to maintain its bioactivity has been demonstrated in clinical studies to lead to significant gains in muscle mass. The age-related loss of lean muscle mass, a condition known as sarcopenia is a major public health problem in the United States and worldwide with an estimated healthcare burden of $18.5 billion in 2000. Recently, it has been demonstrated that derangements in skeletal myocyte mitochondrial function is a major factor contributing to sarcopenia. Sarcopenia is associated with a great risk in sustaining injury due to a fall, which can lead to life threatening complications in the case of the elderly. In a prospective cohort study involving 8,762 men between the ages of 20 and 80, it was found that muscular strength was inversely and independently associated with death from all causes and cancer in men, even after adjustments had been made for cardiorespiratory fitness. See Valko, Marian, et al. Chemico-biological interactions 160.1 (2006): 1-40.

In another nonlimiting embodiment, the egg yolk powder comprises an avian follistatin such as described in U.S. Published Patent Application No. 2007/0275036, the disclosure of which is incorporated herein by reference in its entirety and/or other proteins and/or lipids found in avian eggs and which are beneficial in growth and development of lean muscle tissue.

In another embodiment, the egg yolk powder comprises a spray dried egg yolk powder or one or more proteins and/or lipids derived from egg yolk such as described in U.S. patent application Ser. No. 16/151,601, the disclosure of which is incorporated herein by reference in its entirety.

Nicotinamide adenine dinucleotide (NAD+) is a coenzyme in mammalian cells that plays a key role in metabolism within the mitochondria. Recently, it has come to light that the mitochondria play a much broader function beyond facilitating the conversion of nutrients into energy. With a decline in levels of NAD+, the number and density of mitochondria decrease, resulting in less efficiency and in some cases mitochondrial dysfunction. Dysfunctions of the mitochondria have now been linked to over 40 major diseases and health problems including, but not limited to type 2 diabetes, cancer, Alzheimer's disease and other neurodegenerative diseases. The restoration of NAD+ through supplementation with NAD+ precursors can significantly improve these age-associated functional defects, counteracting many aging diseases.

By combining egg yolk powder with a NAD+ precursor in accordance with the present invention, a synergistic effect can be obtained that will lead to even greater gains in muscle mass and function. Improvements in muscle mass and function lead to better overall health, improved quality of life and enhanced longevity.

Nonlimiting examples of NAD+ precursors which can be administered in accordance with the present invention include nicotinic acid (also referred to as niacin, nicotinamide and nicotinamide riboside. In one nonlimiting embodiment, the NAD+ precursor is nicotinamide riboside. Toxicology studies have shown that adverse effects related to nicotinamide riboside were absent at a dose of 300 mg/kg/day.

In some nonlimiting embodiments, the present invention comprises administration of alternative or additional agents to the NAD+ precursors that promote mitochondrial rejuvenation such as, but in no way limited to, extracts derived from pomegranate.

Oxidative stress is also well recognized as a major contributing factor to the progression of many major diseases including, but not limited to cancer, COPD, type II diabetes and rheumatoid arthritis. In the late 1990s, much excitement was generated due to research highlighting the anti-oxidative properties of resveratrol, a stilbenoid found in red wine. In recent years much interest has developed around pterostilbene, an analog of resveratrol with powerful anti-oxidative properties. The anti-oxidative properties of pterostilbene have been studied in animal models for type II diabetes, Alzheimer's Disease and liver disease.

In the present invention, the anti-oxidative properties of pterostilbene are combined with egg yolk powder to produce synergistic effects relating to better overall health, improved quality of life and enhanced longevity in mammals.

In one nonlimiting embodiment, the method of the present invention comprises administering the egg yolk powder and the NAD+ precursor and/or pterostilbene separately.

In another nonlimiting embodiment, the method involves administering a composition comprising both egg yolk powder and the NAD+ precursor and/or pterostilbene.

In some nonlimiting embodiments, the present invention comprises administration of alternative or additional agents to the NAD+ precursors that promote mitochondrial rejuvenation such as, but in no way limited to extracts derived from pomegranate. In these nonlimiting embodiment, the agent promoting mitochondrial rejuvenation may be administered separated or may be included in the composition with the egg yolk powder. In some nonlimiting embodiments, the composition may further the NAD+ precursor and/or pterostilbene.

In one nonlimiting embodiment, the composition is administered orally on a daily basis, one, two or three times a day.

In one nonlimiting embodiment, the amount egg powder administered is in the range of 0.1 g/day to 100 g/day, or the like. In one nonlimiting embodiment, the composition is administered orally on a daily basis in an amount so that egg yolk powder ranges from about 3 to about 25 grams/day, about 6.6 to about 19.8 grams/day or about 40 to about 300 mg/kg/day, about 80 to about 250 mg/kg/day in humans. In canines, a composition comprising egg yolk powder such as FORTETROPIN is administered orally on a daily basis in an amount ranging from about 200-1000 mg/kg/day or about 300 to about 900 mg/kg/day.

In some embodiments, the composition is administered to provide 300 mg/kg of egg yolk powder daily (one scoop (6600 mg)/22 kg), or the like or a suitable dosage for the weight and characteristics of the mammal.

Preferred is that the egg yolk powder be administered in an amount effective to upregulate mTor pathway activity, downregulate ubiquitin proteasome pathway activity, downregulate serum myostatin levels and/or reduce ActRIIB expression.

The amount of NAD+ precursors, preferably nicotinamide riboside, administered may be in the range of 0.025 g/day to 2.5 g/day, or the like.

The amount of pterostilbene administered may be in the range of 0.005 g/day to 0.500 g/day.

Compositions may also include an effective amount of an agent promoting mitochondrial rejuvenation.

The mixture of egg powder with NAD+ precursor and/or pterostilbene and/or agent promoting mitochondrial rejuvenation can be formulated as tablets, capsules, protein powders or incorporated into protein bars, protein shakes, ice cream and/or ice cream sandwiches, yogurt and/or frozen yogurt for oral administration, and/or the like. All methods and/or routes of administration suitable for administration of the compositions disclosed herein are contemplated by and within embodiments of the present disclosure.

In exemplary embodiments, an egg yolk powder in combination with the NAD+ precursor and/or pterostilbene and/or agent promoting mitochondrial rejuvenation, consistent with the present disclosure, can be combined and/or mixed with various other elements or ingredients prior to administration that may cause a synergistic beneficial health benefit. For example, an egg yolk powder composition consistent with the present disclosure can be mixed with coconut oil, coconut butter or coconut powder. An egg yolk powder composition consistent with the present disclosure can also be mixed with a combination of nuts including almonds, Brazil nuts, cashews, chestnuts, hazelnuts, macadamia nuts, peanuts, pecans, pine nuts and/or walnuts, or the like. An egg yolk powder composition consistent with the present disclosure can also be mixed with combinations of seeds including chia seeds, flax seeds, sesame seeds and/or pumpkin seeds, or the like. An egg yolk powder composition consistent with the present disclosure can also be mixed with avocado powder, or the like. An egg yolk powder composition consistent with the present disclosure can also be mixed with cheese powder, or the like. An egg yolk powder composition consistent with the present disclosure can also be mixed with sweeteners including stevia, sucralose, erythritol, xylitol, monk fruit and/or agave nectar, or the like. Any and all combinations of the ingredients listed above and similar ingredients are contemplated by and

Further, dosages of egg yolk powder, NAD+ precursor and/or pterostilbene and/or agent promoting mitochondrial rejuvenation may be modified for efficacy, for example, may be administered at a higher or lower dosage or administered more than once daily or less than once daily. Mammals may be dosed to the closest 4 scoop, or the like, without underdosing. The composition may be formed in a powder that may be mixed with other food to facilitate ingestion.

However, as will be understood by the skilled artisan upon reading this disclosure, the compositions described herein can be formulated for administration to a subject via any conventional means including, but not limited to, oral, or buccal.

Moreover, the compositions described herein, can be formulated into any suitable dosage form, including but not limited to, aqueous oral dispersions, liquids, gels, syrups, elixirs, slurries, suspensions and the like, for oral ingestion by an individual in need, solid oral dosage forms, controlled release formulations, fast melt formulations, effervescent formulations, lyophilized formulations, tablets, powders, pills, dragees, capsules, delayed release formulations, aqueous liquid dispersions, self-emulsifying dispersions, solid solutions, liposomal dispersions, solid dosage forms, powders, tablets, capsules, pills, delayed release formulations.

Formulations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including glucose, fructose, lactose, sucrose, mannitol, sorbitol, stevia extract, or sucralose; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Formulations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, pellets, granules. In other embodiments, the pharmaceutical formulation is in the form of a powder. In still other embodiments, the pharmaceutical formulation is in the form of a tablet. Additionally, formulations described herein may be administered as a single capsule or in multiple capsule dosage form. In some embodiments, the formulation is administered in two, or three, or four, capsules or tablets.

Soft gel or soft gelatin capsules may be prepared, for example, without limitation, by dispersing the formulation in an appropriate vehicle (vegetable oils are commonly used) to form a high viscosity mixture. This mixture is then encapsulated with a gelatin-based film using technology and machinery known to those in the soft gel industry. The industrial units so formed are then dried to constant weight.

In some embodiments, the formulations may include other medicinal or pharmaceutical agents, carriers, diluents, dispersing agents, suspending agents, thickening agents, adjuvants, such as preserving, stabilizing, wetting or emulsifying agents, solution promoters, and/or buffers. In addition, the formulations can also contain other therapeutically valuable substances.

The formulations described herein can include egg yolk powder and NAD+ precursor and/or pterostilbene and/or an agent promoting mitochondrial rejuvenation and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination(s) thereof. In still other aspects, using standard coating procedures, a film coating is provided around the formulation of the compound described herein. In one embodiment, some or all of the particles of the compound described herein are coated. In another embodiment, some or all of the particles of the compound described herein are microencapsulated. In still another embodiment, the particles of the compound described herein are not microencapsulated and are uncoated.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Formulations including egg yolk powder and NAD+ precursor and/or pterostilbene and/or an agent promoting mitochondrial rejuvenation, as described herein, may be manufactured in a conventional manner, such as, by way of example only, by means of conventional mixing, dissolving, granulating, dragee-making, levigating, emulsifying, encapsulating, entrapping or compression processes.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

Binders imparting cohesive qualities may also be used. Examples include, but are not limited to, alginic acid and salts thereof; cellulose derivatives such as carboxymethylcellulose, methylcellulose, hydroxypropylmethylcellulose, hydroxyethylcellulose, hydroxypropylcellulose, ethylcellulose, and microcrystalline cellulose; microcrystalline dextrose; amylose; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose, glucose, dextrose, molasses, mannitol, sorbitol, xylitol, and lactose; a natural or synthetic gum such as acacia, tragacanth, ghatti gum, mucilage of isapol husks, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder.

Formulators skilled in art can determine the binder level for the formulations, but binder usage level of up to 70% in tablet formulations is common.

Compositions may further comprise carriers of relatively nontoxic chemical compounds or agents that facilitate the incorporation of a compound into cells or tissues. Nonlimiting examples include binders, suspending agents, disintegration agents, filling agents, surfactants, solubilizers, stabilizers, lubricants, wetting agents, diluents, and the like. Suitable carriers for use in solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Dispersing agents and/or viscosity modulating agents include materials that control the diffusion and homogeneity of a compound through liquid media or a granulation method or blend method. In some embodiments, these agents also facilitate the effectiveness of a coating or eroding matrix. Nonlimiting examples of diffusion facilitators/dispersing agents include hydrophilic polymers, electrolytes, a Tween, PEG, polyvinylpyrrolidone, and carbohydrate-based dispersing agents such as hydroxypropyl celluloses (e.g., HPC, HPC-SL, and HPC-L), hydroxypropyl methylcelluloses (e.g., HPMC K100, HPMC K4M, HPMC Kl5M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers, block copolymers of ethylene oxide and propylene oxide; and poloxamines, tetrafunctional block copolymers derived from sequential addition of propylene oxide and ethylene oxide to ethylenediamine, polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents that are particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

Compositions of the present invention may further comprise diluents used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose; dibasic calcium phosphate, dicalcium phosphate dihydrate; tricalcium phosphate, calcium phosphate; anhydrous lactose, spray-dried lactose; pregelatinized starch, compressible sugar; mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; sodium chloride; inositol, bentonite, and the like.

Compositions may further comprise an enteric coating, a substance that remains substantially intact in the stomach but dissolves and releases the egg yolk powder in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

In addition, the compositions may comprise an erosion facilitator, a material that controls the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

Filling agents including compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like can also be included in the compositions. Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In addition, flavoring agents and/or sweeteners can be used in the compositions and may include acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate, maltol, mannitol, maple, marshmallow, menthol, mint cream, mixed berry, neohesperidine DC, neotame, orange, pear, peach, peppermint, peppermint cream, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

The compositions may further comprise lubricants and/or glidants that prevent, reduce or inhibit adhesion or friction of materials. Nonlimiting examples of lubricants include stearic acid, calcium hydroxide, talc, sodium stearyl fumerate, a hydrocarbon such as mineral oil, or hydrogenated vegetable oil such as hydrogenated soybean oil, higher fatty acids and their alkali-metal and alkaline earth metal salts, such as aluminum, calcium, magnesium, zinc, stearic acid, sodium stearates, glycerol, talc, waxes, boric acid, sodium benzoate, sodium acetate, sodium chloride, leucine, a polyethylene glycol (e.g., PEG-4000) or a methoxypolyethylene glycol, sodium oleate, sodium benzoate, glyceryl behenate, polyethylene glycol, magnesium or sodium lauryl sulfate, colloidal silica, a starch such as corn starch, silicone oil, a surfactant, and the like.

Plasticizers, compounds used to soften the microencapsulation material or film coatings to make them less brittle may also be included in the compositions. Examples of suitable plasticizers include, but are not limited to, polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

The compositions may further comprise solubilizers such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

In addition, the compositions may comprise stabilizers such as antioxidation agents, buffers, acids, preservatives and the like.

Suitable suspending agents for use in solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxypropylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants including compounds such as sodium lauryl sulfate, sodium docusate, Tweens, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide and the like may also be included. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

Viscosity enhancing agents including, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof may also be included.

In addition, wetting agents including compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like may be included in these compositions.

In some embodiments, solid dosage forms, e.g., tablets, capsules, are prepared by mixing the egg yolk powder and NAD+ precursor and/or pterostilbene as described herein, with one or more pharmaceutical excipients to form a bulk blend composition. When referring to these bulk blend compositions as homogeneous, it is meant that the particles of egg yolk powder and NAD+ precursor and/or pterostilbene and/or agent that promotes mitochondrial rejuvenation are dispersed evenly throughout the composition so that the composition may be readily subdivided into equally effective unit dosage forms, such as tablets, pills, and capsules.

Conventional techniques include, e.g., one or a combination of methods: (1) dry mixing, (2) direct compression, (3) milling, (4) dry or non-aqueous granulation, (5) wet granulation, or (6) fusion. See, e.g., Lachman et al., “The Theory and Practice of Industrial Pharmacy” (1986).

It should be appreciated that there is considerable overlap between additives used in the solid dosage forms described herein. Thus, the above-listed additives should be taken as merely exemplary, and not limiting, of the types of additives that can be included.

A capsule may be prepared, for example, by placing the bulk blend of the formulation of the compound described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials which sufficiently isolate the compound from other non-compatible excipients. Materials compatible with the egg yolk powder are those that delay the release of the egg yolk powder in vivo.

In other embodiments, the formulations described herein, which include the egg yolk powder and NAD+ precursor and/or pterostilbene and/or an agent promoting mitochondrial rejuvenation, are solid dispersions. Methods of producing such solid dispersions are known in the art and include, but are not limited to, for example, U.S. Pat. Nos. 4,343,789, 5,340,591, 5,456,923, 5,700,485, 5,723,269, and U.S. Pub. Appl 2004/0013734.

In still other embodiments, the formulations described herein are solid solutions. Solid solutions incorporate a substance together with the active agent and other excipients such that heating the mixture results in dissolution of the drug and the resulting composition is then cooled to provide a solid blend which can be further formulated or directly added to a capsule or compressed into a tablet. Methods of producing such solid solutions are known in the art and include, but are not limited to, for example, U.S. Pat. Nos. 4,151,273, 5,281,420, and 6,083,518.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components. The enteric coated oral dosage form may also be a capsule (coated or uncoated) containing pellets, beads or granules of the solid carrier or the composition.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating for the methods and compositions described herein to achieve delivery to the lower gastrointestinal tract.

In some embodiments, formulations are provided that include particles of egg yolk powder and NAD+ precursor and/or pterostilbene and/or an agent promoting mitochondrial rejuvenation as described herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of egg yolk powder, the liquid dosage forms may include additives, such as: (a) disintegrating agents; (b) dispersing agents; (c) wetting agents; (d) at least one preservative, (e) viscosity enhancing agents, (t) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

In one nonlimiting embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the formulations described herein can be self-emulsifying drug delivery systems (SEDDS). Emulsions are dispersions of one immiscible phase in another, usually in the form of droplets. Generally, emulsions are created by vigorous mechanical dispersion.

SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563.

Buccal formulations that include egg yolk powder and NAD+ precursor and/or pterostilbene and/or an agent that promotes mitochondrial rejuvenation may be administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with the egg yolk powder, and any other components that may be present in the buccal dosage unit. Generally, the polymeric carrier comprises hydrophilic (water-soluble and water-swellable) polymers that adhere to the wet surface of the buccal mucosa. Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

In certain embodiments, delivery systems for pharmaceutical compounds may be employed, such as, for example, liposomes and emulsions. In certain embodiments, compositions provided herein can also include a mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

1: A method for enhancing overall health and longevity in a mammal, said method comprising administering to the mammal a composition comprising egg yolk powder in combination with a NAD+ precursor and/or pterostilbene, wherein the egg yolk powder is administered in an amount effective to upregulate mTor pathway activity, downregulate ubiquitin proteasome pathway activity, downregulate serum myostatin levels and/or reduce ActRIIB expression.

2: The method of claim 1 wherein the composition comprises a fertilized egg yolk derived product.

3: The method of claim 2 wherein the fertilized egg yolk derived product is FORTETROPIN.

4: The method of claim 1 wherein the composition comprises avian follistatin.

5: The method of claim 1 wherein the mammal is administered a composition comprising egg yolk powder and a NAD+ precursor and/or pterostilbene.

6: The method of claim 1 wherein the NAP+ precursor is nicotinamide riboside.

7: The method of claim 1 wherein the mammal is a human.

8: The method of claim 1 comprising administering an agent promoting mitochondrial rejuvenation.

9: The method of claim 8 wherein administration of the composition comprising egg yolk powder in combination with a NAD+ precursor and/or pterostilbene and/or an agent promoting mitochondrial rejuvenation promotes mitochondrial health, gains in muscle thickness and/or protection against oxidative damage in the mammal.

10: A composition for enhancing overall health and longevity in mammals, said composition comprising egg yolk powder and a NAD+ precursor and/or pterostilbene and/or an agent promoting mitochondrial rejuvenation wherein the egg yolk powder is administered in an amount effective to upregulate mTor pathway activity, downregulate ubiquitin proteasome pathway activity, downregulate serum myostatin levels and/or reduce ActRIIB expression.

11: The composition of claim 10 wherein the composition comprises a fertilized egg yolk derived product.

12: The composition of claim 11 wherein the fertilized egg yolk derived product is FORTETROPIN.

13: The composition of claim 10 wherein the composition comprises avian follistatin.

14: The composition of claim 10 wherein the NAP+ precursor is nicotinamide riboside.

15: The composition of claim 10 wherein the agent promoting mitochondrial rejuvenation is a pomegranate extract.
